(19) United States
(12) Patent Application Publication
(10) Pub. No.: US 2026/0191565 A1
(43) Pub. Date: Jul. 9, 2026

(54) LOAD-CARRYING ROD

(71) Applicant: SPINESAVE AG, Embrach (CH)

(72) Inventor: Stefan FREUDIGER, Bremgarten (CH)

(21) Appl. No.: 19/128,203

(22) PCT Filed: Nov. 8, 2023
(86) PCT No.: PCT/EP2023/081200

§ 371 Date: May 8, 2025

(30) Foreign Application Priority Data
Nov. 8, 2022 (EP) 22206227.5

(51) Int. Cl. A61B 17/70; A61B 17/00; B33Y 70/00; B33Y 80/00

(52) U.S. Cl. CPC A61B 17/7002; B33Y 70/00; B33Y 80/00; A61B 2017/00526

(57) ABSTRACT

A load-carrying rod for an implant system has a structure of individual interconnected solidified droplets so that the transitions from one droplet to another act as crack stoppers in the event of a crack.

The present invention relates to a load carrying rod as part of an implant system according to the preamble of claim 1.

According to the state of the art, rods for implant systems are for example forged, cast, pressed, drawn, injection-molded, extruded, or rolled, which generally results in largely homogeneous cross-sections with largely similar properties. At most direction-dependent differences, so-called anisotropy, can be achieved with these methods. This does not refer to rods that are designed of different materials, such as composite materials, which are individually designed from a fiber and a matrix, for example. In the present case, it refers to bodies which are designed from one and the same type of material, wherein the material may have different properties within its type, such as the mechanical stiffness.

Accordingly, the invention described below addresses the problem of providing arrangements with the aid of which a crack on the surface of a load-carrying rod cannot simply pass through a homogeneous cross-section.

US2013/0158606 A1 discloses a rod of this kind. It is typically manufactured by injection-molding. Consequently, the rod has a homogeneous composition, in which cracks propagate rapidly depending on the tension applied and its duration.

An object of the invention is to propose a rod as part of an implant system having improved resistance against crack propagation.

Such a rod is defined in claim 1. The further claims define preferred embodiments and manufacturing methods of such a rod.

The solution to this problem is characterized by the fact that the rod cross-section is built-up by substructures which form boundary surfaces between one another, so that the mechanical properties within the substructure differ from the properties at the transitions.

Preferably, such a rod has a cross-section that is not homogeneous, so that a possible crack on the surface cannot grow through the entire cross-section, but encounters areas that have uneven crack growth properties, since the interior of the droplets is held together more by cohesive forces and the transitions are held together more by adhesive forces.

The present invention discloses features by means of which a crack on the surface of a rod is prevented from propagating further through the cross-section.

These substructures consist of solidified droplets strung together and on top of each other, so that the properties within the droplet are preferably different from the properties at the transitions from one droplet to another. In this case, the droplets are strung together with sufficient energy in such a way that they connect to one another by melting. The droplet diameter is 0.01 to 1 millimeter, preferably 0.2 to 1.0 millimeter. Such a structure can be created by the known 3D printing as described for example in EP1886793 B1, where discontinuous droplets are deposited sequentially.

The droplets can also be spherical in shape.

Biocompatible aromatic polycarbonate urethane is preferably used as the material.

The present invention is explained by mean of preferred embodiments with reference to Figures. The Figures schematically show:

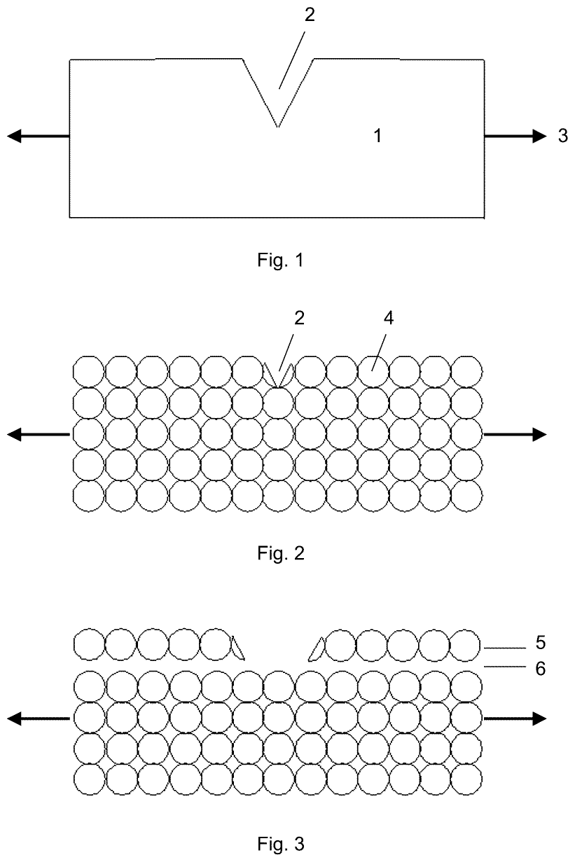

FIG. 1 A schematic illustration of a rod 1 with a homogeneous cross-section, in which a crack 2 (enlarged) in the sense of a tensile elongation fracture propagates through the entire cross-section under an axial load 3 with only little resistance to further cracking.

FIG. 2 A schematic illustration (heavily enlarged) of a rod 1 with a structure of many droplets 4, in which a crack 2 can indeed pass through a droplet, but where it is stopped when it reaches the boundary to the next droplet. Droplet here is a schematic designation of the initial shape during manufacturing. The final shape is then more like a cube.

FIG. 3 A schematic illustration (heavily enlarged) of a rod 1 after a crack of an outer layer 5 with subsequent delamination 6 of the outer layer.

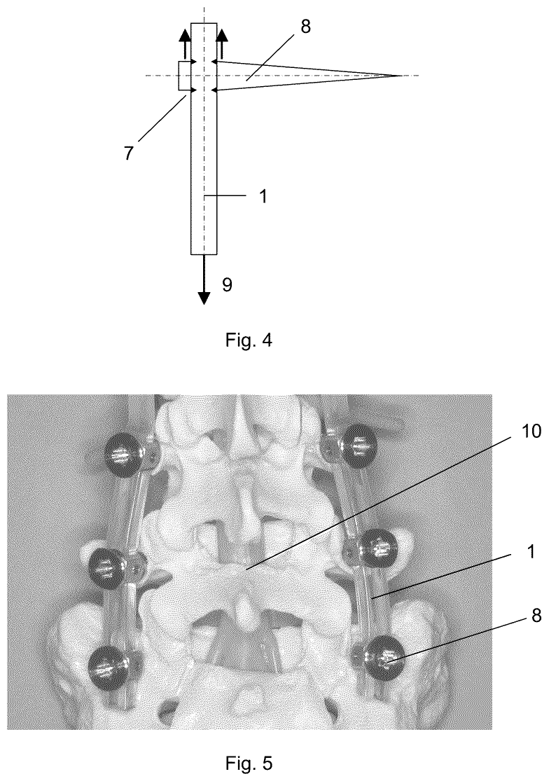

FIG. 4 A rod 1 with a schematically shown clamping 7 in a pedicle screw 8. The rod is subject to a longitudinal force 9 which is transferred by the clamping in the pedicle screw.

FIG. 5 A picture of a rod 1 with its pedicle screws 8 as an implant system for the spinal column 10.

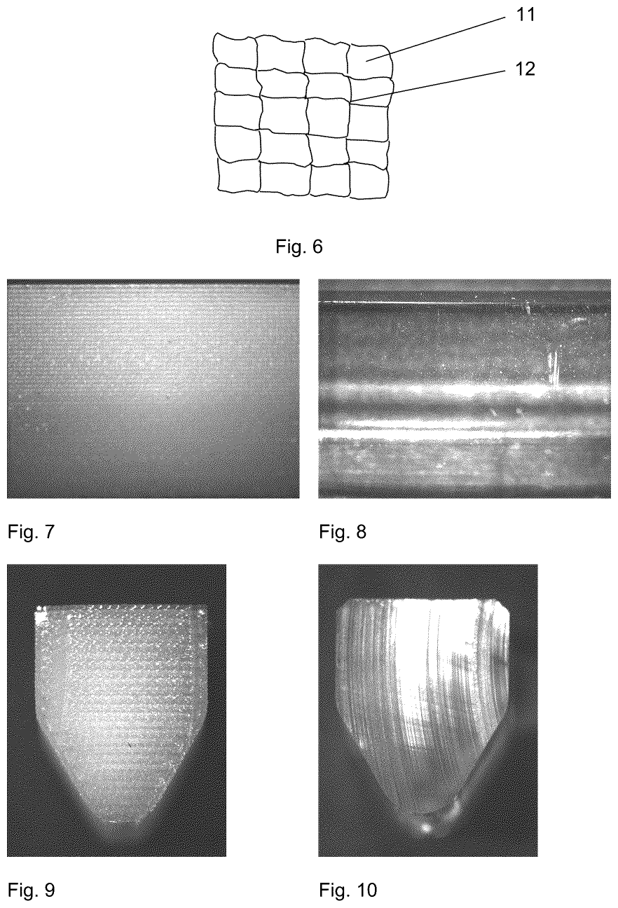

FIG. 6 A schematic illustration (heavily enlarged) of a rod section in a more realistic representation, in which the droplets are deformed into bodies 11, which are melted all around with the neighbors 12.

FIG. 7 A picture of a surface of a rod according to the invention.

FIG. 8 A picture of a surface of a prior art rod.

FIG. 9 A picture of the surface of a transverse cut through a rod according to the invention.

FIG. 10 A picture of the surface of a transverse cut through a prior art rod.

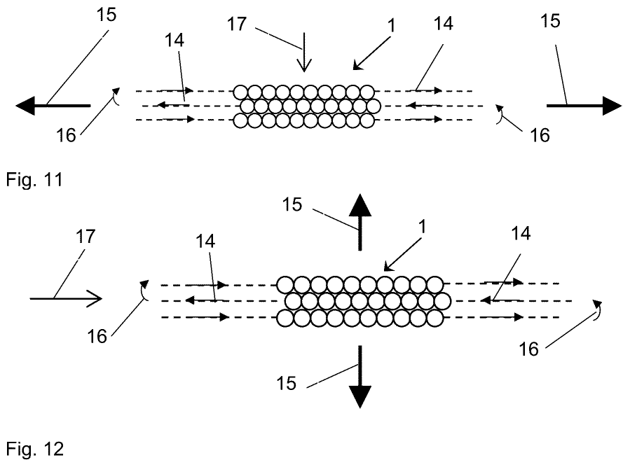

FIG. 11 Schematic illustration of the effect of deposition along tension.

FIG. 12 Schematic illustration of effect of deposition transverse to tension.

A precaution against a crack growth transverse to the longitudinal direction or length of a rod is important because a crack transverse to the longitudinal direction can occur for various reasons, such as short-duration overstressing. On the one hand, as shown in FIG. 5, rods are often clamped transversely to the longitudinal direction in pedicle screws for load transfer, so that together with the forces to be transmitted in the longitudinal direction, a multiaxial stress state occurs which, because of its tendency to brittle fracture, favors the initiation of cracks. On the other hand, rods can also be subjected to bending stresses, so that a crack can also occur on the tensile side (outside of the bend) if the local strength is exceeded or if there may be geometric defects or material defects on the surface.

If a crack occurs on a rod surface, this crack can propagate through an entire cross-section under continued stress and result in a separation of the rod. This crack propagation can occur more easily in a homogeneous cross-section than in a heterogeneous cross-section, where the physical properties change across the cross-section and thus locally place areas with higher resistance to growth in the path of the crack.

The crack tip always also means a notch which favors the crack propagation. Since the notch is changed at each transition of a phase boundary in a heterogeneous cross-section, the crack growth is made more difficult.

If a crack is prevented from growing further, it can lead to delamination of the cracked layer, which indeed reduces the overall strength of the rod, but ideally can still ensure a sufficient residual strength.

More particularly, clamping in a pedicle screw is preferably realized by a rib surrounding the rod (cf. EP2309936A). The rib, as shown in FIG. 4 as clamping 7, imposes pressure along a circumferential line. In such a clamping, it is particularly advantageous that the rod has a high resistance against initiation of cracks, and that cracks once initiated do not propagate.

In 3D printing in particular, depending on the material used, the droplets connect to one another by intermolecular forces, supported by entanglements in the sense of microscopic form-locking, and partly also by chemical bonds. The different thermal history can also lead to different mechanical behavior between the interior and the surface of a droplet.

In developing the present invention, rods have been produced using a 3D manufacturing process, where droplets of building material are deposited one after the other and adjacent to the immediately precedingly deposited droplet. Preferably, the deposition is performed along a meander course (cf. direction of deposition 14 and deposition reversal 16 for a meandering deposition), as indicated in FIGS. 11 and 12. Thereby, layers of lines of droplets are produced. The layers are produced one on top of the other and yielding the rod 1. Preferably, the building material is a thermoplastic polymeric material.

In FIGS. 11 and 12, tension 15 applied to the rod 1 in use is normally directed in longitudinal direction of the rod 1, or parallel to its length. A clamping force is exerted by the clamping device 7 (FIG. 4) and, in contrast to tension 15, is locally concentrated to a narrow area, ideally a line, on the surface of rod 1.

In FIG. 11, deposition of droplets (arrows 14) occurred in parallel to expected tension 15, yet transverse to crack initiation 17 by a clamping force. Hence, a deposited droplet is each time deposited adjacent to a droplet immediately precedingly deposited and, therefore, in a less solidified state compared with the droplets of the preceding line. It is assumed that the droplets forming a line and are deposited one after the other are more tightly interconnected than with the neighboring droplet of adjacent lines. The effect exists as well between layers of lines of droplets. Globally, the rods present a higher resistance to tension in the deposition direction 14 as a consequence thereof, than transverse thereto, as set forth below. Furthermore, if the clamping force exerted by the clamping 7 leads to a crack initiation 17 and a crack 2. Where a crack 2 splits a line of droplets, the propagating crack encounters the zone of less tight connection between the split line and the neighboring line. Thereafter, it encounters once again a line of more tightly interconnected droplets. By this change of physical properties, the propagation of the crack 2 is effectively inhibited and eventually stopped.

It may be supposed, too, that the less tight connection between lines of droplets 14 reduces or avoids that the splitting of one line of droplets imposes a splitting force on the neighboring line in direction of propagation of a crack, so that the clamping, crack initiation 17 hits a fresh, not prestressed line of droplets. On the other hand, a line of droplets located more to the interior are more and more supported in their resistance against splitting by their environment, i.e. the adjacent lines of droplets.

The situation with the crack initiation 17 in direction of the lines of droplets 14 and tension transverse thereto is illustrated in FIG. 12. The tension 15 tends to separate the lines of droplets from the adjacent lines of droplets. As the adhesion between two lines of droplets is lower than between droplets in a line, the rod 1 tends to delaminate along planes between the layers of lines droplets, the resistance against the crack initiation 17 is reduced, and the resistance against propagation of cracks is impaired. Understandably, resistance against tension 15 is lower than with lines of droplets oriented in direction of applied tension 15 in FIG. 11.

It is conceivable that instead of a meandering deposition, i.e. reversing (arrows 16) the direction of the progress (arrows 14) of the deposition of droplets, the lines of droplets are deposited in the same direction without reversing yet with returning after finishing a line to the start of the next line of droplets. This manufacturing method may be slower than meandering as the droplet deposition means needs to be moved back over the full length of a line of droplets to the starting point, yet the droplets in neighboring lines are deposited with an about constant difference in time hence the adhesion between neighboring lines is expected to be about constant over the length of the lines of droplets.

As well, the layers of lines of droplets may be produced in a meandering fashion which is preferred in view of reduced production time, yet alternatively each layer may be produced in the same direction, and the production means returned to the starting zone of the preceding layer. In returning to the start zone, the deposition time gap between droplets between two consecutive layers is constant and the adhesion between layers is expected to be more constant over the plane of the layers than in meandering.

In a fatigue test (repetitive tension and compression loads), the following was observed: A crack may be initiated at the location where the rod is clamped in the screw head 7 due to the local multiaxial stress state which may develop brittleness. In an injection molded rod (state-of-the-art) such a crack may propagate across the entire cross section and may lead to a rod failure. In contrast, in a 3D printed rod according to the present invention, such crack not only may be initiated at much higher displacements (e.g. 10% to 50%) but may also propagate through the outermost layers only.

FIGS. 7 to 10 demonstrate that the described production process yields a rod obviously different from the rods obtained by the manufacturing process according to the prior art. Generally, known rods are either directly produced by bulk production like injection molding or extrusion, or are cut from a piece of raw material obtained by such manufacturing process. Consequently, the rods according to the prior art are continuous. The face of a cut (FIG. 10) through such a rod as well as its surface (FIG. 8) show a continuous, smooth and mirror-like reflecting appearance.

In contrast, a rod 1 according to the present invention shows a face of a cut (FIG. 9) as well as a surface (FIG. 7) which have a non-continuous appearance reminding of tightly arranged minute bodies and merely disperses light instead of reflecting it.

Typical parameters of a rod 1 are:
The preferred length of a rod 1 is 2 to 50 cm.
The load range that a rod 1 must repeatedly withstand during its implantation period (i.e. while it is implanted) is maximally 800 N tensile force and maximally 400 N compressive force. Preferably, it complies with both limits.
A rod 1 must withstand a static tensile force of maximally 1600 N and/or a static compression force of maximally 800 N.
The maximum shear deformability is 8 mm for a 30 mm long specimen.
Materials with preferred flexural moduli of 50 to 250 N/mm² in a conditioned/implantable state are used.
The longitudinal stiffness of a rod 1 is in the range from minimally 50 N/mm to maximally 1000 N/mm.

The typical parameters or properties set forth above are to be understood that most preferably, a rod 1 complies with all these properties, yet it may as well show only a part of it down to only one of the typical properties.

Structures of similar materials with different mechanical properties can also be built-up by means of the 3D printing method. One possibility of the advantageous oblique transition is shown in EP2869773 (B1). However, a welded connection has the disadvantage that the transition of stiffness is limited to the welding plane, whereas this can be done continuously with 3D printing.

From the previously described embodiment, variations of the invention are conceivable by the person skilled in the art without departing from the scope of protection which is defined by the claims. Conceivable is, for example:
Manufacturing a rod of the structure described by another method than the one presented, even a non-3D printing method.

1. A load-carrying rod as part of an implant system for the dynamic stabilization of the spinal column, wherein the rod has an inhomogeneous cross-sectional structure of droplets strung together, stacked on each other and interconnected with adjacent droplets in order to inhibit the propagation of a crack.

2. The load-carrying rod according to claim 1, wherein the rod is manufactured in 3D printing by additive manufacturing, preferably by a 3D manufacturing process where droplets are deposited sequentially and adjacently in lines.

3. The load-carrying rod according to claim 1, wherein the rod is made of a polycarbonate urethane.

4. The load-carrying rod according to claim 1, wherein the rod is made from polycarbonate urethane of different mechanical stiffnesses in individual areas.

5. The load-carrying rod according to claim 1, wherein the droplet size is 10 to 1000 micrometer, preferably 200 to 1000 micrometer.

6. The load-carrying rod according to claim 1, wherein the boundary surfaces between droplets have different physical properties than the interior of the droplets and thus prevent a mechanical crack from propagating.

7. The load-carrying rod according to claim 1, wherein the difference in physical properties inside and outside the droplets is due to cohesive forces and adhesive forces.

8. The load-carrying rod according to claim 1, wherein the failure stress in a longitudinal direction is greater than transverse to the longitudinal direction.

9. The load-carrying according to claim 1, wherein the rod has at least one of the following properties:
length of 2 to 50 centimeter;
resistant to a repeated application of a tensile force of at most 800 N;
resistant to a repeated application of compressive force of at most 400 N;
resistant to a static tensile force of maximally 1600 N;
resistant to a static compression force of maximally 800 N;
maximum shear deformation of 8 mm for a length of 30 mm;
flexural modulus of at least 50 N/mm² and at most 250 N/mm²;
longitudinal stiffness is minimally 50 N/mm and maximally 1000 N/mm.

10. A manufacturing method for manufacturing the rod according claim 1, wherein droplets are deposited in lines with one droplet adjacent to another, so that the adjacent droplets are at least superficially intermixed, and to let the droplet solidify.

11. The method of claim 0, wherein the droplets in a line are adjacently and immediately consecutively deposited in parallel to the length of the load-carrying rod.
